(19) United States
(12) Patent Application Publication
(10) Pub. No.: US 2009/0233845 A1
(43) Pub. Date: Sep. 17, 2009

(54) Methods and compositions for non-covalently enhanced receptor binding

(76) Inventors: Soumya Ray, Cambridge, MA (US); Marc Charette, Needham, MA (US); Seth P. Finklestein, Needham, MA (US)

Correspondence Address: CLARK & ELBING LLP, 101 FEDERAL STREET, BOSTON, MA, 02110, US

(21) Appl. No.: 12/319,261

(22) Filed: Jan. 5, 2009

Related U.S. Application Data
(60) Provisional application No. 61/010,007, filed on Jan. 4, 2008.

(51) Int. Cl. A61K 38/22; C07K 14/505; C07K 14/50; A61P 9/10

(52) U.S. Cl. 514/8; 530/395; 530/397

(57) ABSTRACT

The invention features contacting (in vitro or in vivo) a receptor-binding ligand with an organic molecule, which can be a small molecule (i.e., an organic molecule that is not a peptide), or a peptide that noncovalently binds to the ligand and either another ligand for the receptor (either a second copy of the first ligand, or a second, different ligand), the receptor itself, or both. Exemplary ligand/receptor pairs include FGF-2/FGF-R1 and EPO/EPO-R. The invention further features pharmaceutical compositions and methods of using such compositions for treating various medical conditions.

### CROSS-REFERENCE TO RELATED APPLICATIONS

This application claims priority to U.S. Provisional Application No. 61/010,007, filed Jan. 4, 2008, which is hereby incorporated by reference.

### BACKGROUND

Many ligands bind to their receptors as dimers. Examples include basic fibroblast growth factor (bFGF, FGF-2), transforming growth factor beta (TGF-β), erythropoietin (EPO), granulocyte colony stimulating factor (G-CSF), and growth hormone (GH). In some cases, it has been demonstrated that covalent linking together of monomers to form dimers enhances ligand binding to receptors and consequent signal transduction. Several patent applications describe such covalently-linked ligand dimers.

### SUMMARY OF THE INVENTION

Rather than creating enhanced binding dimerized ligands in which the ligand monomers are covalently bonded, we produce enhanced receptor binding by non-covalent bond formation, for example, Van der Waals forces or electrostatic interactions, between an organic molecule and one or more ligand monomers and, in some instances, another molecule involved in receptor-mediated cellular processes, including the receptor itself. The organic molecule-ligand interaction results in increased ligand/receptor “on time,” enhancing signal transduction and increasing biological/therapeutic activity.

Accordingly, in general, the invention features contacting (in vitro or in vivo) a receptor-binding ligand with an organic molecule, which can be a small molecule (i.e., an organic molecule that is not a peptide), or a peptide that noncovalently binds to the ligand and either another ligand for the receptor (either a second copy of the first ligand, or a second, different ligand), the receptor itself, or both.

In one aspect of the invention, it features a method of enhancing the binding of a first ligand to a cellular receptor by contacting the first ligand with an exogenous organic molecule that non-covalently binds to the first ligand and non-covalently binds to either (i) a second ligand for the receptor (which may or may not be the same as the first ligand) or (ii) the receptor itself. The contacting may be carried out in vitro, in vivo, or in silico.

In a related aspect, the invention features a method of treating a medical condition in a human patient by administering to the patient, in an amount sufficient to treat the medical condition, an organic compound that non-covalently binds to a first ligand for a cellular receptor and non-covalently binds to either (i) a second ligand for the receptor (which may or may not be the same as the first ligand) or (ii) the receptor itself to enhance the binding of the first ligand to the receptor. Exemplary medical conditions include cerebrovascular, peripheral vascular, and cardiovascular diseases and anemia.

The invention further features a method of identifying an organic molecule that enhances binding of a first ligand to a cellular receptor by determining in silico whether the organic molecule non-covalently binds to the first ligand and to either (i) a second ligand for the receptor (which may or may not be the same as the first ligand) or (ii) the receptor itself, resulting in enhanced binding of the first ligand to the receptor. In this method, the organic molecule may be selected from a library based on its chemical and physical properties, such as one or more of: the presence of hydrogen bond donors, the presence of hydrogen bond acceptors, molecular weight, elemental composition, solubility, reactivity, stability, toxicity, and lipophilicity. The organic molecule may also includes only elements selected from C, O, N, S, P, F, Cl, Br, I, B, Na, K, Mg, and Ca.

The organic molecules can be used to manufacture ligand-containing complexes to be administered to patients, or, more preferably, they can be administered to patients (preferably orally) so that noncovalent binding, e.g., of FGF-2 or EPO, occurs within the patient's body following administration. Accordingly, the invention features a pharmaceutical composition including a first ligand for a cellular receptor and/or an organic molecule that non-covalently binds to the first ligand and either (i) a second ligand for the receptor (which may or may not be the same as the first ligand) or (ii) the receptor itself to enhance the binding of the first ligand to the receptor; and (c) a pharmaceutically acceptable carrier. In addition, the invention features a pharmaceutical composition including a pharmaceutically effective amount of a compound of FIG. 3, 4, 12, or 13, together with a pharmaceutically acceptable carrier.

The invention also features a tetramer of four molecules of EPO, each of which is noncovalently bound to two other EPO molecules. The tetramer may further include two exogenous organic molecules that stabilize the formation of noncovalent bonds between two or more EPO molecules. Two sets of two molecules of EPO are preferably noncovalently bound via Ala-1, Pro-2, Pro-3, Arg-4, Leu-5, Ile-6, Cys-7, Asp-8, Cys-161, Arg-162, Thr-163, Gly-164, Asp-166, and/or Arg-167. Any exogenous organic molecule present in the tetramer may noncovalently bind to one or more of these residues in each of two molecules of EPO to enhance binding of the two EPO molecules.

The first ligand is, for example, a peptide (fewer than thirty amino acids), a polypeptide (between thirty-one and one hundred amino acids), or a protein (more than one hundred amino acids). In preferred embodiments, the first ligand is FGF-2 or EPO, and the receptor is FGF-R1 or EPO-R. Exemplary sites of interaction between two or more ligands and/or a ligand and its receptor include (i) Asn-27, Arg-120, Thr-121, Lys-125, Lys-129, Gln-134, Lys-135, and Ala-136 of FGF-2; and Glu-159, Lys-160, Lys-163, Lys-172, Thr-173, Phe-176, Lys-177, Lys-207, Val-208, Arg-209, Thr-212, Ile-216, Met-217, Asp-218, and Ser-219 of FGF-R1; (ii) Arg-97, Leu-98, Glu-99, Ser-100, Asn-101, and Asn-102 of FGF-2; and Pro-169, Ala-170, Ala-171, Asp-217, Ser-218, Val-219, Val-220, Pro-221, Ser-222, Asp-223, Val-248, Glu-249, Arg-250, and Ser-251 of FGF-R1; (iii) Ala-1, Pro-2, Pro-3, Arg-4, Leu-5, Ile-6, Cys-7, Asp-8, Cys-161, Arg-162, Thr-163, Gly-164, Asp-166, and Arg-167 in each of two molecules of EPO; (iv) Thr-148, Pro-149, Met-150, Thr-151, Ser-152, His-153, Arg-154, Leu-175, Glu-176, Gly-177, and Arg-178 of EPO-R; and Asp-8, Ser-9, Arg-10, Val-11, Leu-12, Glu-13, Arg-14, Tyr-15, Leu-16, Leu-17, Glu-18, Ala-19, Lys-20, Glu-21, Ala-22, Glu-23, and Lys-24 of EPO. Organic molecules of the invention preferably bind to at least one of these residues on each of the ligands and/or ligand and receptor. In other embodiments, the organic molecule binds to at least 2, 3, 4, 5 or more residues between two ligands or ligand and receptor. Organic molecules may also bind to multiple molecules of a receptor as well. As will understood, biological variability between subjects may lead to the absence of or a change to one of the residues identified above. The invention encompasses use of organic molecules to enhance receptor binding of FGF-2 and EPO in all biological forms.

In particular embodiments, the organic molecule decreases the off-rate of the ligand/receptor binding by a factor of 2, 5, 10, 100, or more compared to the off-rate in the absence of the organic molecule. Alternatively, or in addition, the organic molecule binds to the ligand and/or receptor with a strength of 5-200 kJ/mol. In other embodiments, the organic molecule is not a peptide and has a molecular weight between 150 and 5,000.

Other features and advantages will be apparent from the following description, the drawings, and the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a depiction of FGF-2/FGF-R1 Site I; organic molecules may bind simultaneously to 2 FGF-2 ligands and 2 FGF-R1 receptor molecules at this site.

FIG. 2 is a depiction of FGF-2/FGF-R1 Site II; organic molecules may bind simultaneously to 2 FGF-2 ligands and 2 FGF-R1 receptor molecules at this site.

FIG. 3 is a table of organic molecules identified in silico as capable of binding to Site I of FIG. 1.

FIG. 4 is a table of organic molecules identified in silico as capable of binding to Site II of FIG. 2.

FIG. 5 is a depiction of organic molecules binding to Site I of FIG. 1. As can be seen, the molecules share general structural features.

FIG. 6 is a depiction of organic molecules binding to Site II of FIG. 2. As can be seen, the molecules share general structural features.

FIG. 7 is a depiction of an example molecule bound to Site I of FIG. 1.

FIG. 8 is a depiction of an example molecule bound to Site II of FIG. 2.

FIG. 9 is a depiction of EPO/EPO-R Site I; organic molecules may bind simultaneously to 2 EPO molecules.

FIG. 10 is a depiction of EPO/EPO-R Site II; organic molecules may bind simultaneously to 1 EPO molecule and 2 EPO-R receptor molecules.

FIG. 11 is a depiction of a noncovalent tetramer of EPO.

FIG. 12 is a table of organic molecules identified in silico as capable of binding to Site I of FIG. 9.

FIG. 13 is a table of organic molecules identified in silico as capable of binding to Site II of FIG. 10.

FIG. 14 is a depiction of organic molecules binding to Site I of FIG. 9. As can be seen, the molecules share general structural features.

FIG. 15 is a depiction of organic molecules binding to Site II of FIG. 10. As can be seen, the molecules share general structural features.

FIG. 16 is a depiction of an example molecule bound to Site I of FIG. 9.

FIG. 17 is a depiction of an example molecule bound to Site II of FIG. 10.

FIG. 18 is a graph showing the increase in FGF-2 activity in the presence of increasing concentrations of heparin.

### DETAILED DESCRIPTION OF THE INVENTION

In general, the invention provides methods and compositions wherein an organic molecule enhances the binding of a ligand to its receptor. Enhancement may occur, e.g., by the organic molecule noncovalently binding to multiple ligands (either the same or different) to form dimers or larger polymers, e.g., tetramers, or by the organic molecule noncovalently binding to the ligand (or multiple ligands) and the receptor (or multiple receptors). Combinations of these types of noncovalent interactions are also included in the invention.

The ability of organic molecules (sometimes referred to herein as compounds) to bind to a particular ligand can be determined using the procedures and assays described herein. Many of the individual compounds and classes of compounds described herein have been extensively studied, and compounds can either be purchased commercially or synthesized using procedures well known in the art of organic chemistry. Organic molecules of the invention may also be designed in silico. The compounds are selected or designed to bind to sites at the interface of two ligand molecules and/or a ligand and its receptor. The practitioner skilled in the art will appreciate that there are a number of ways to select or design compounds of the present invention, see, e.g., U.S. 2006/0194821. This design or selection may begin with selection of the various moieties that fill interfacial binding sites.

There are a number of ways to select moieties to fill individual interfacial binding sites. These include visual inspection of a physical model or computer model of the active site and manual docking of models of selected moieties into various binding sites. Modeling software that is well known and available in the art may also be used. These include QUANTA (Molecular Simulations, Inc., Burlington, Mass.), and SYBYL (Molecular Modeling Software, Tripos Associates, Inc., St. Louis, Mo.). This modeling step may be followed by energy minimization with standard molecular mechanics forcefields such as CHARMM and AMBER (AMBER: Weiner, et al., J. Am. Chem. Soc. 106:765 (1984); CHARMM: Brooks, et al., Comp. Chem. 4:187 (1983)).

In addition, there are a number of more specialized computer programs to assist in the process of optimally placing either complete molecules or molecular fragments into the binding site. These include: GRID (Goodford, J. Med. Chem. 28:849-857 (1985), available from Oxford University, Oxford, UK); MCSS (Miranker, et al., Proteins: Structure, Function and Genetics 11, 29-34 (1991), available from Molecular Simulations, Burlington, Mass.); and DOCK (Kuntz, et al., J. Mol. Biol. 161:269-288 (1982), DOCK is available from the University of California, San Francisco, Calif.).

Once suitable binding orientations have been selected, complete molecules can be chosen for biological evaluation. In the case of molecular fragments, they can be assembled into a compound. This assembly may be accomplished by connecting the various moieties to a central scaffold. The assembly process may, for example, be done by visual inspection followed by model building, again using software such as Quanta or Sybyl. A number of other programs may also be used to help select ways to connect various fragments. These include: CAVEAT (Bartlett, et al. In Molecular Recognition in Chemical and Biological Problems, Special Pub., Royal Chem. Soc. 78: 182-196 (1989)), 3D Database systems such as MACCS-3D (MDL Information Systems, San Leandro, Calif., Martin (J. Med. Chem. 35: 2145 (1992)), and HOOK (Molecular Simulations, Burlington, Mass.).

In addition to the above computer-assisted modeling of compounds, the compounds of this invention may be constructed “de novo” using an empty active site. Such methods are well known in the art. They include, for example: LUDI (Bohm, J. Comp. Aid. Molec. Design. 6:61-78 (1992), LUDI is available from Biosym Technologies, San Diego, Calif.), LEGEND (Nishibata, Tetrahedron 47:8985 (1991), LEGEND is available from Molecular Simulations, Burlington, Mass.), and LeapFrog (available from Tripos Associates, St. Louis, Mo.). A number of techniques commonly used for modeling drugs may be employed (e.g., Cohen, et al., J. Med. Chem. 33:883 (1990); Navia, et al., Current Opinions in Structural Biology 2:202 (1992); Baldwin, et al., (J. Med. Chem. 32:2510 (1989); Appelt, et al., J. Med. Chem. 34:1925 (1991); and Ealick, et al., Proc. Nat. Acad. Sci. USA 88, 11540 (1991)).

A variety of conventional techniques may be used to carry out each of the above evaluations as well as the evaluations necessary in screening a candidate compound for activity in enhancing ligand-receptor binding. Generally, these techniques involve determining the location and binding proximity of a given moiety, the occupied space of a bound compound, the amount of complementary contact surface between the compound and ligand/receptor, an estimate of the deformation energy generated in the binding of the compound to the receptor, and an estimate of hydrogen bonding strength and/or electrostatic interaction energies produced by compound/receptor binding. Examples of known disciplines useful in the above evaluations include: quantum mechanics, molecular mechanics, molecular dynamics, Monte Carlo sampling, systematic searches, and distance geometry methods (Marshall, Ann. Rev: Pharmacol. Toxicol. 27:193 (1987)). Computer software has been developed for use in in silico screening of compounds for the above-described properties. Examples include: Gaussian (M. J. Frisch, Gaussian, Inc., Pittsburgh, Pa.,); AMBER; QUANTA/CHARMM (Molecular Simulations, Inc., Burlington, Mass.); Insight II/Discover (Biosysm Technologies Inc., San Diego, Calif.); and Schrodinger First Discovery Suite with GLIDE (Schrödinger, Inc., Portland, Oreg.).

Different classes of compounds, according to this invention, may interact in similar ways with the various binding regions of ligand-ligand and/or ligand-receptor binding sites. Different classes of compounds of this invention may also use different scaffolds or core structures, any of which will allow the necessary moieties to be placed in the binding site so that the specific interactions necessary for binding are obtained.

The process by which organic molecules that enhance ligand-receptor binding through non-covalent interactions are identified and used is exemplified below, with respect to two biologically active proteins, fibroblast growth factor-2 (FGF-2) and erythropoietin (EPO).

FGF-2

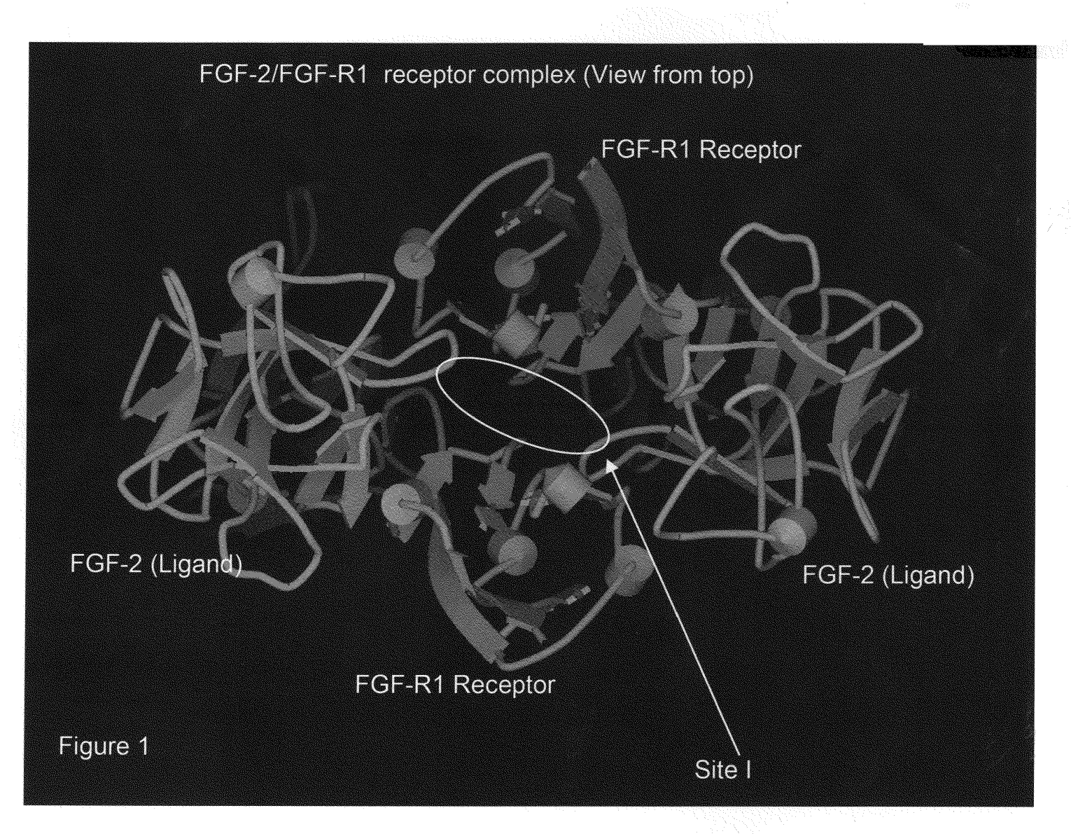

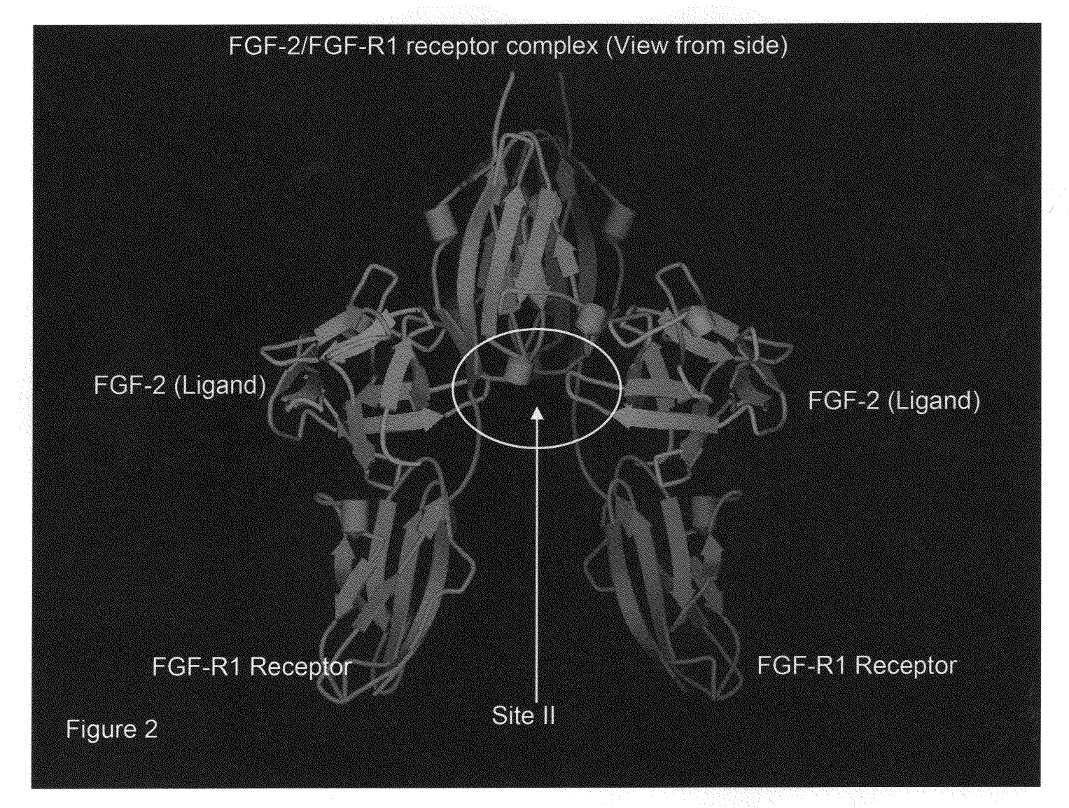

Molecular models of the FGF-2/FGF-2 receptor (FGF-R1) complex were constructed, based on the published crystal structure of this complex (FIGS. 1 and 2) (pdb code 1FQ9; Schlessinger et al. Mol. Cell 6: 743-750 (2000)). These models show a close juxtaposition of FGF-2 monomers when bound to the receptor, and two sites to which organic molecules bind were identified. For Site I (FIG. 1), the following amino acids to which an organic molecule may bind were identified: on FGF-2: Asn-27, Arg-120, Thr-121, Lys-125, Lys-129, Gln-134, Lys-135, Ala-136; and on FGF-R1: Glu-159, Lys-160, Lys-163, Lys-172, Thr-173, Phe-176, Lys-177, Lys-207, Val-208, Arg-209, Thr-212, Ile-216, Met-217, Asp-218, and Ser-219. An organic molecule of the invention preferably binds noncovalently to at least one of these residues on each of an FGF-2 molecule and an FGF-R1 molecule. As is shown in FIG. 1, the organic molecule may simultaneously bind to two molecules of FGF-2 and two molecules of FGF-R1.

For Site II (FIG. 2), the following amino acids to which an organic molecule may bind were identified: on FGF-2: Arg-97, Leu-98, Glu-99, Ser-100, Asn-101, and Asn-102; and on FGF-R1: Pro-169, Ala-170, Ala-171, Asp-217, Ser-218, Val-219, Val-220, Pro-221, Ser-222, Asp-223, Val-248, Glu-249, Arg-250, and Ser-251. An organic molecule of the invention preferably binds noncovalently to at least one of these residues on each of an FGF-2 molecule and an FGF-R1 molecule. As is shown in FIG. 2, the organic molecule may simultaneously bind to two molecules of FGF-2 and two molecules of FGF-R1.

An exemplary method for identifying organic molecules to enhance the binding of FGF-2 to its receptor is as follows. All computations were carried out on a 3.2 Ghz PC running RedHat Linux (Fedora Version 7). We employed the libraries listed in Table 1 for initial identification of organic molecules, and we used property-based filtering to select for molecules that satisfied the “Lipinski Rule of Five” (Adv Drug Del Rev 23: 3-25 (1997)) but with relaxed threshold values (hydrogen bond donors≧5; hydrogen bond acceptors≧10; 10≧molecular weight≧800; ClogP≧7) compared to the original Lipinski values to minimize wasting compounds. After filtering with Lipinski rules, the library was further filtered to remove compounds containing heavy metals; only those compounds that had elements selected from C, O, N, S, P, F, Cl, Br, I, B, Na, K, Mg, and Ca were kept for further processing. The filtered subset was treated with the ligprep module of Schrodinger First Discovery Tool (Schrödinger Inc.) to remove counter ions, adjust charge states, and generate tautomers wherever applicable. The bmin module was used to generate energy minimized structures for all the compounds in each database.

TABLE 1

| | | Number of |
|---|---|---|
| | | compounds used |
| Company | Library name | for docking |
| Asinex | Asinex gold & | 209418 |
| | platinum collection | |
| Key Organics | Bionet | 41000 |
| ChemBridge | Chembridge microformat | 100000 |
| ChemDiv | ChemDiv | 257132 |
| ChemStar | Chemstar | 49179 |
| Enamine | Enamine compound | 287289 |
| | collection | |
| InterBioScreen | IBS | 199363 |
| MayBridge | MaybBridge | 44435 |
| Molecular Diversity | MDPI | 121244 |
| Preservation International | | |
| Pharmeks | Pharmeks Main database | 105600 |
| Prestwick | Prestwick Drug Like | 640 |
| | Molecule collection | |
| NCI/NIH Developmental | NCI | 13267 |
| Therapeutics Program | | |
| Specs and BioSpecs | Compound collection | 240000 |
| Sigma | Rare Chemical Database | 51294 |
| Timtec | Timtec | 160000 |
| Tripos | Tripos | 4621 |
| Zelinsky | | 45892 |

The final database was stored in maestro (Schrödinger Inc. proprietary format). Hydrogen atoms were added to the FGF-R1 receptor structure complexed to FGF-2 (1evt.pdb) and energy minimized. Residue 179 was chosen as the center for mass for generation of the grid for docking calculation.

The program Glide (part of the Schrbdinger First Discovery Suite) was used for docking. The first main docking job was carried out using the default Glide parameters. A maximum of 5000 molecules sorted by glidescore were requested per database for the initial round of docking calculation. The collections obtained from the first round of docking were combined to obtain a total of 32,214 molecules. These molecules were again docked with the “extra-precision” mode of GLIDE, which exacts severe penalties for complexes that violate established principles, such as that charged and strongly polar groups be adequately exposed to solvent. A maximum of 500 poses were requested in this round of docking. To facilitate computation the main job was split into subjobs using the paraglide utility to allow for parallel processing on dual core processor computers.

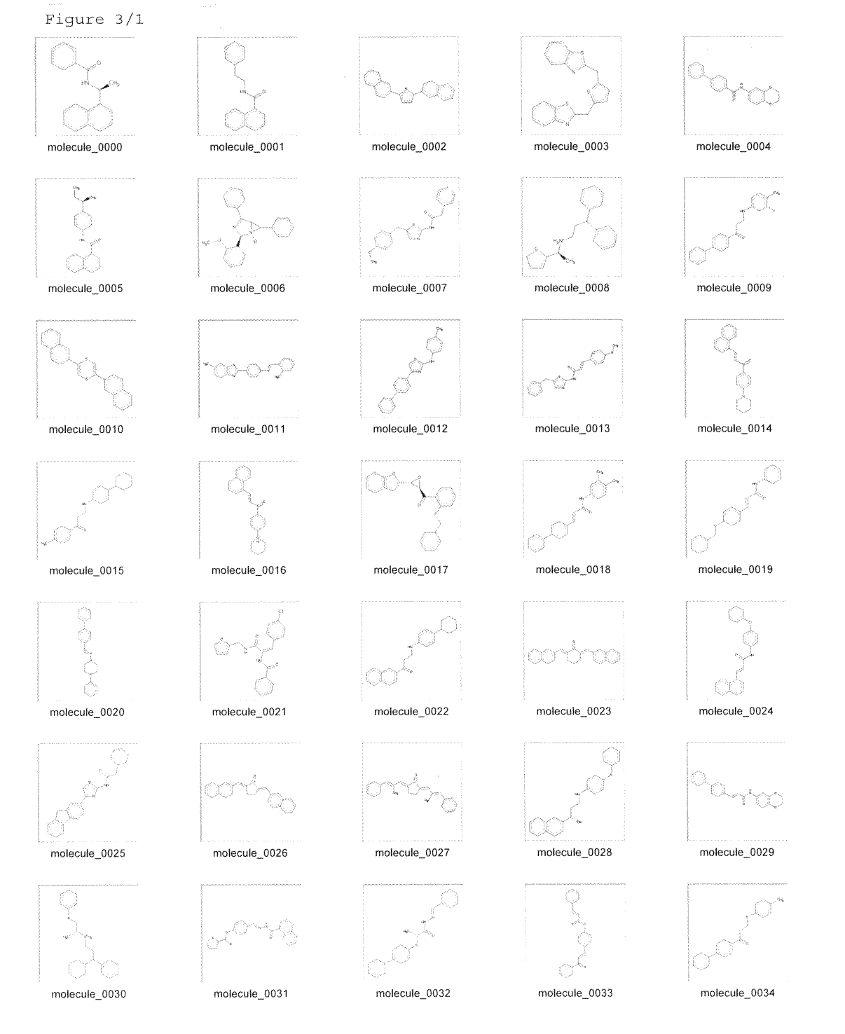

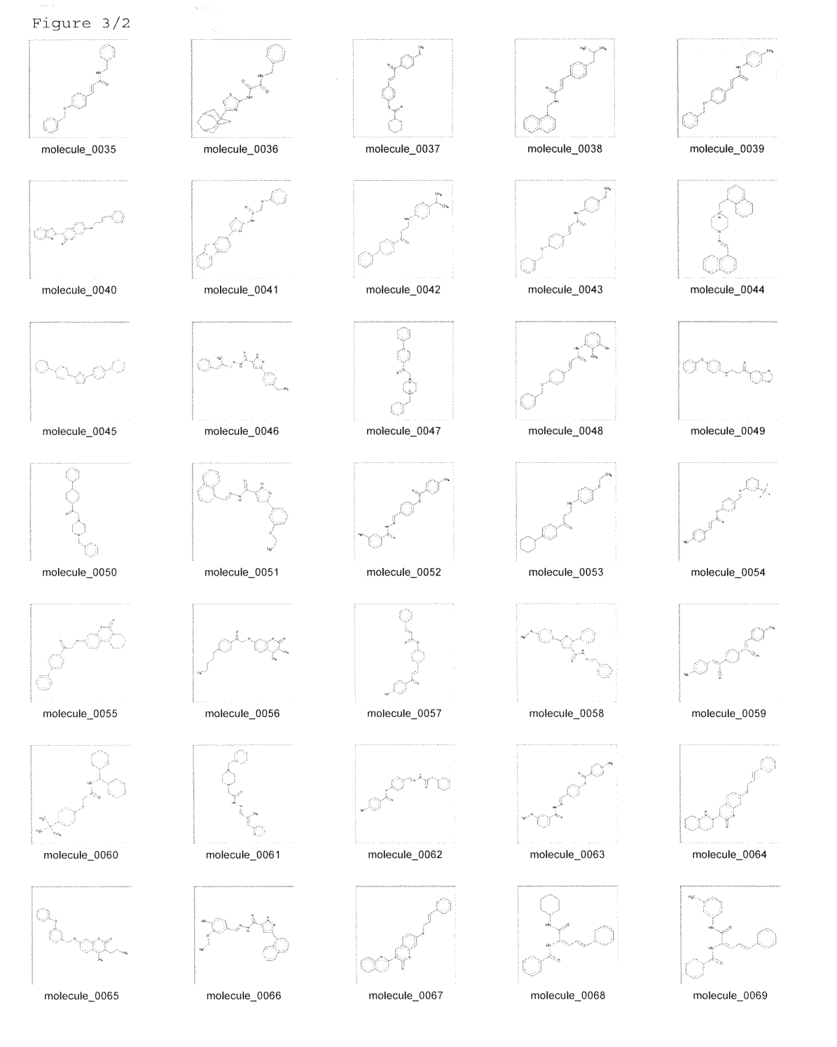

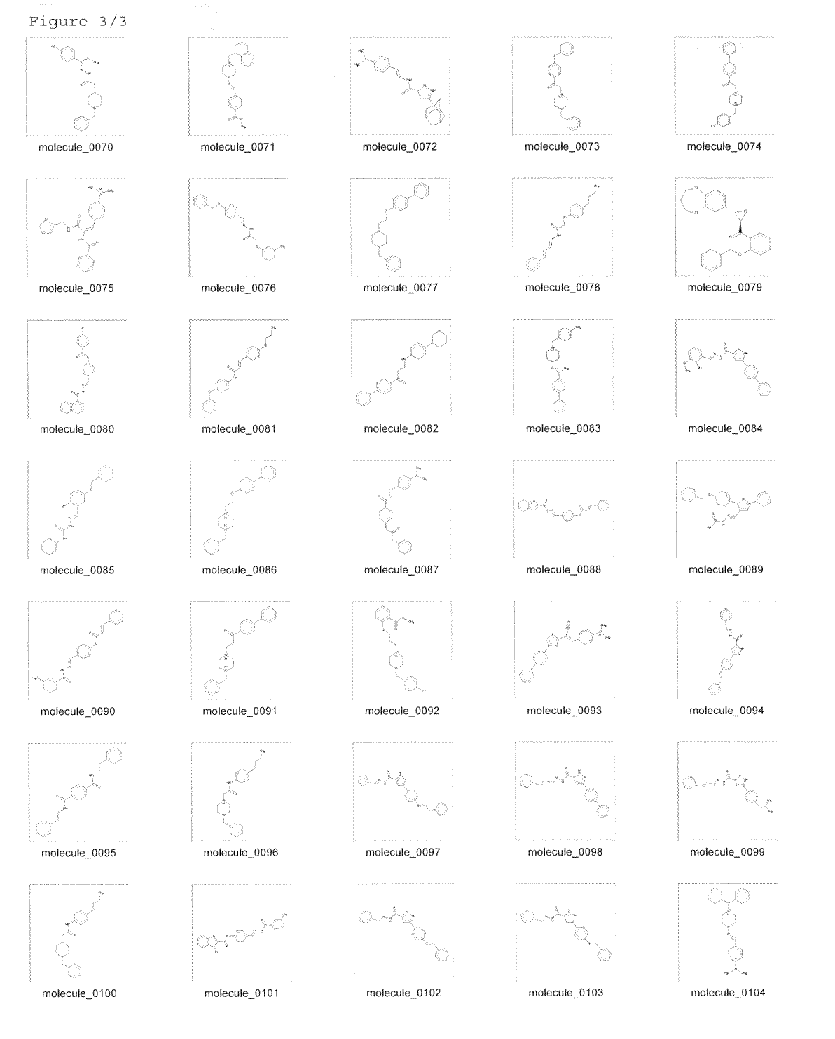

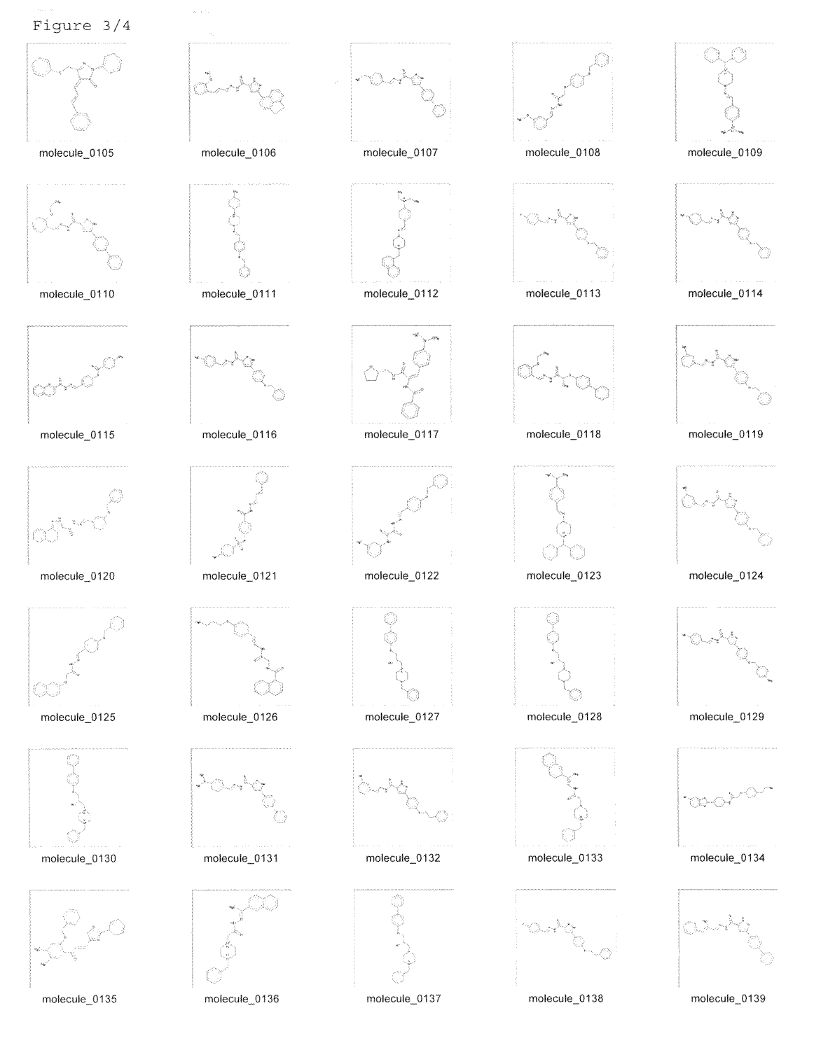

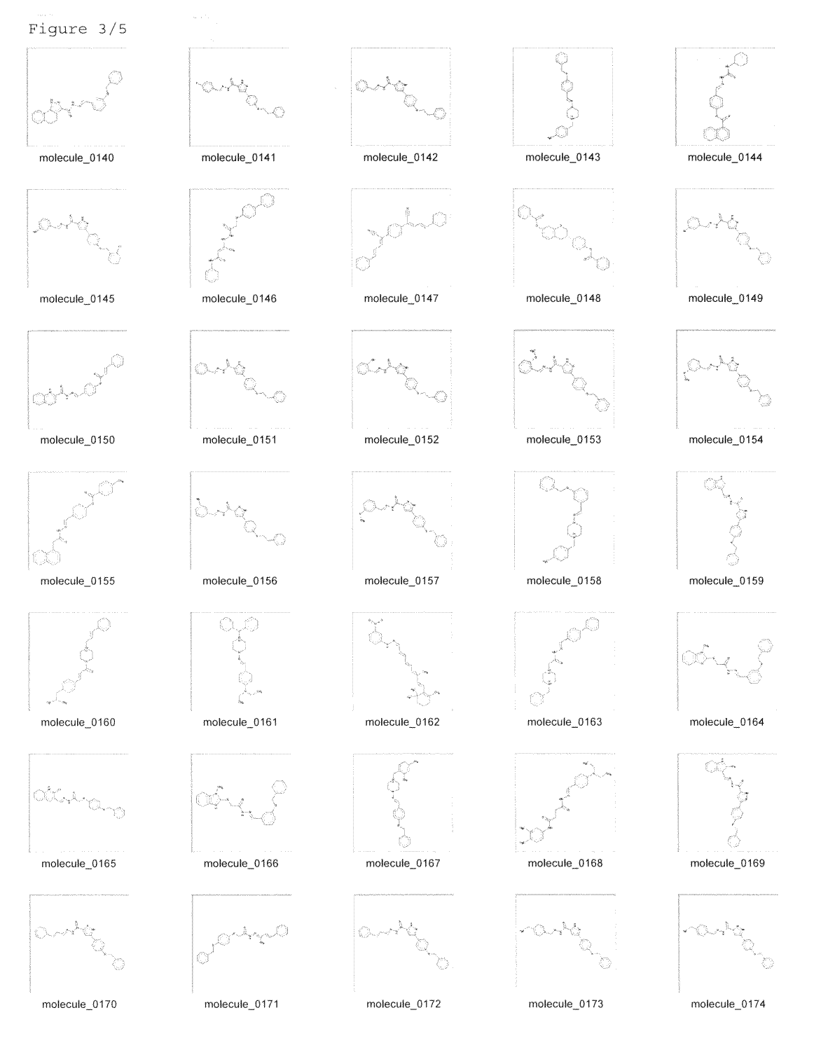

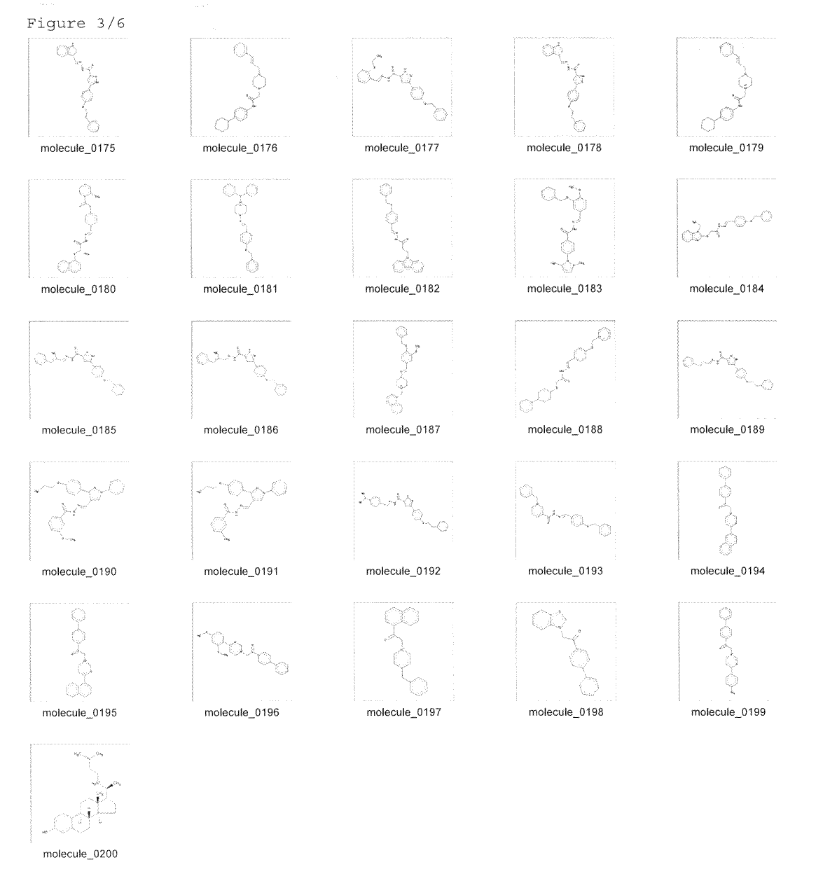

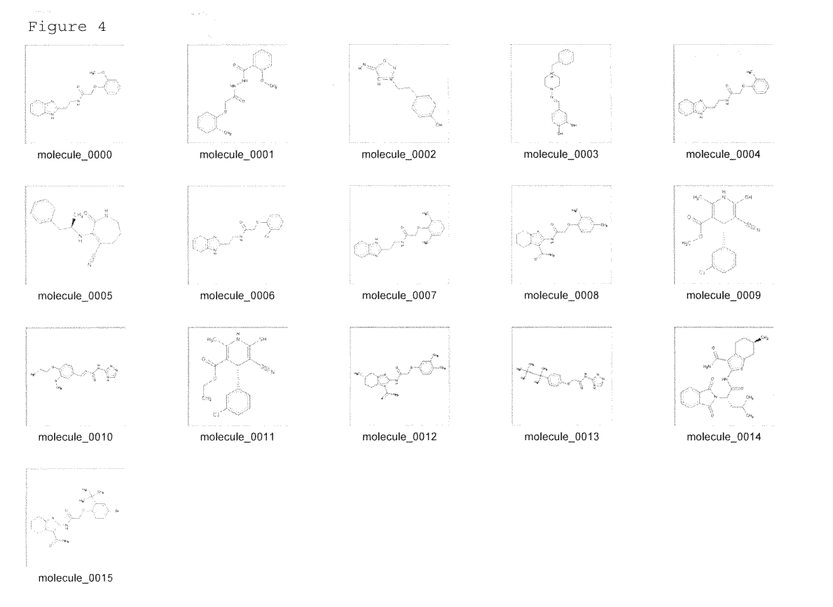

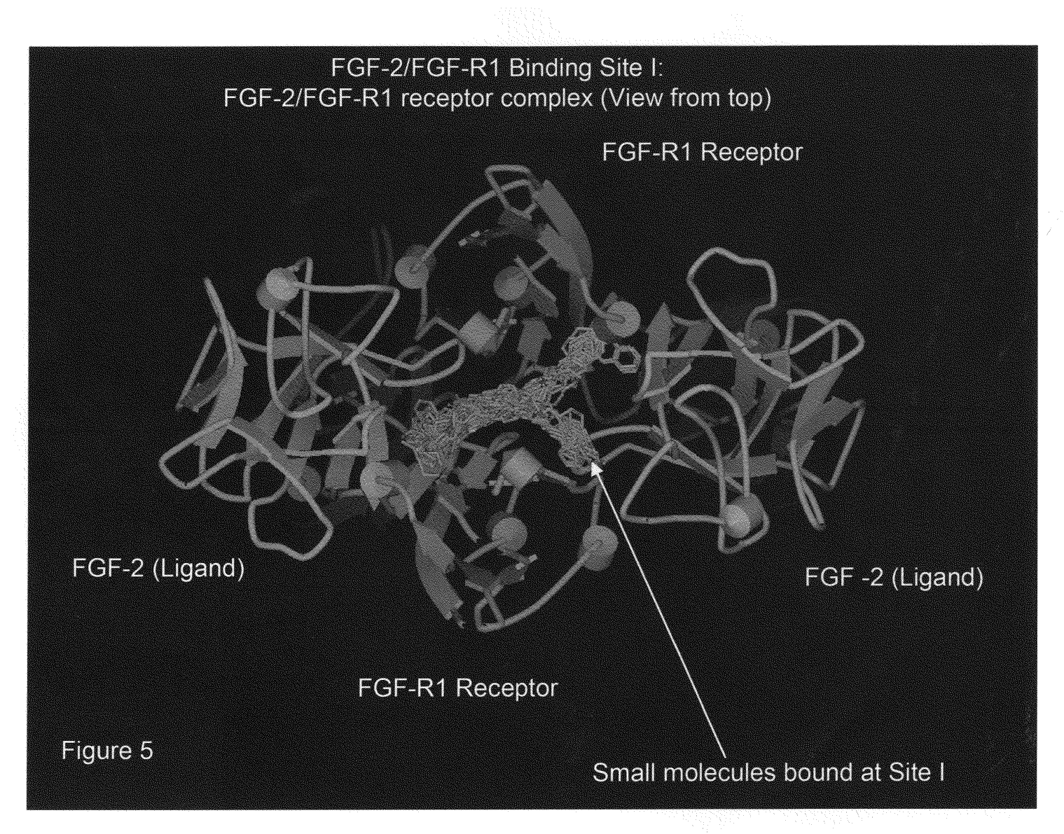

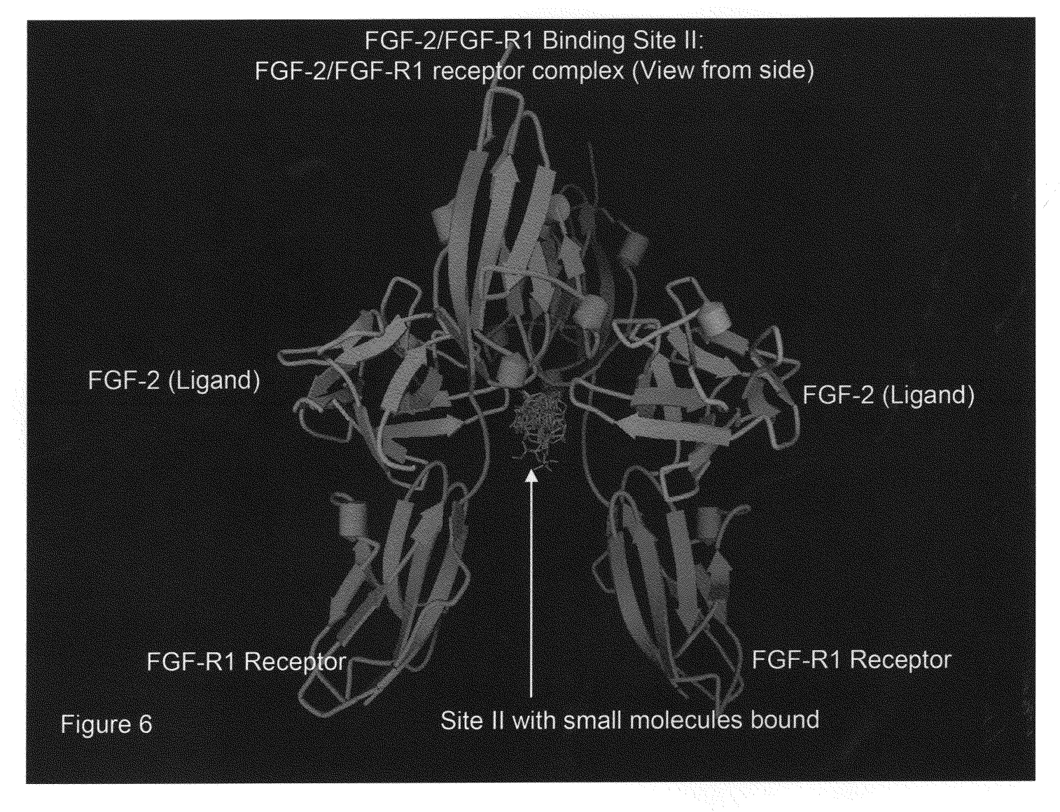

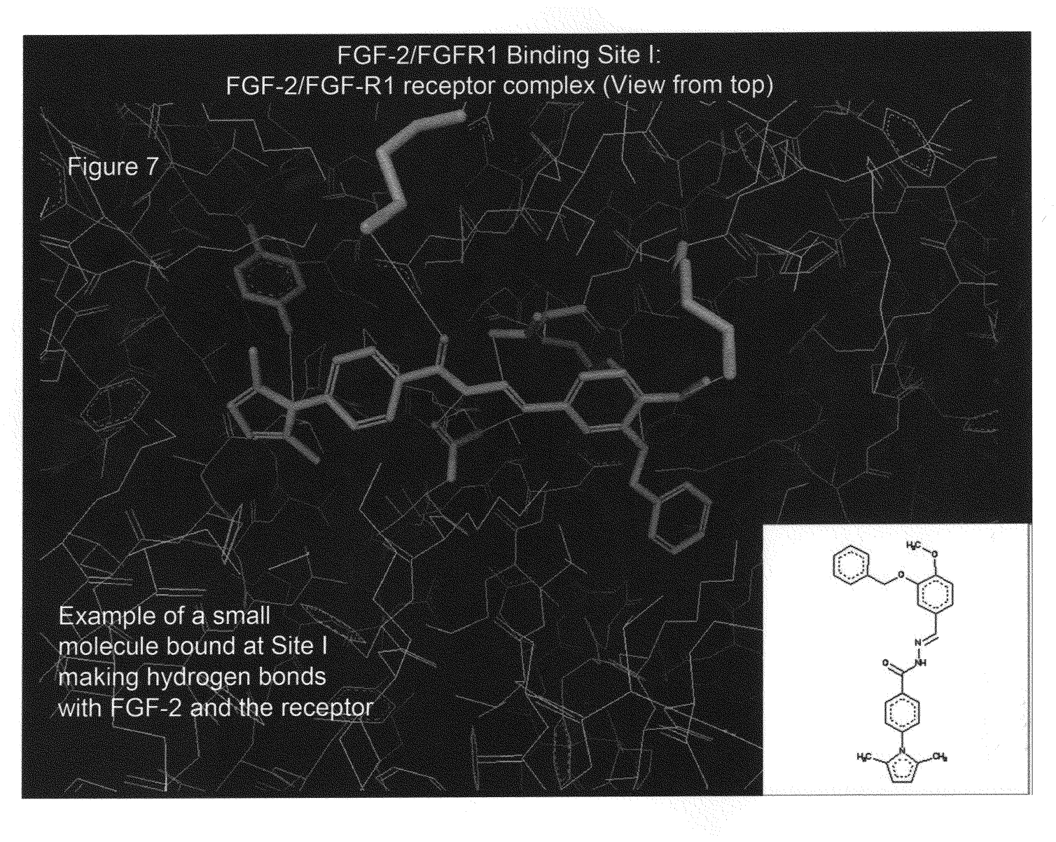

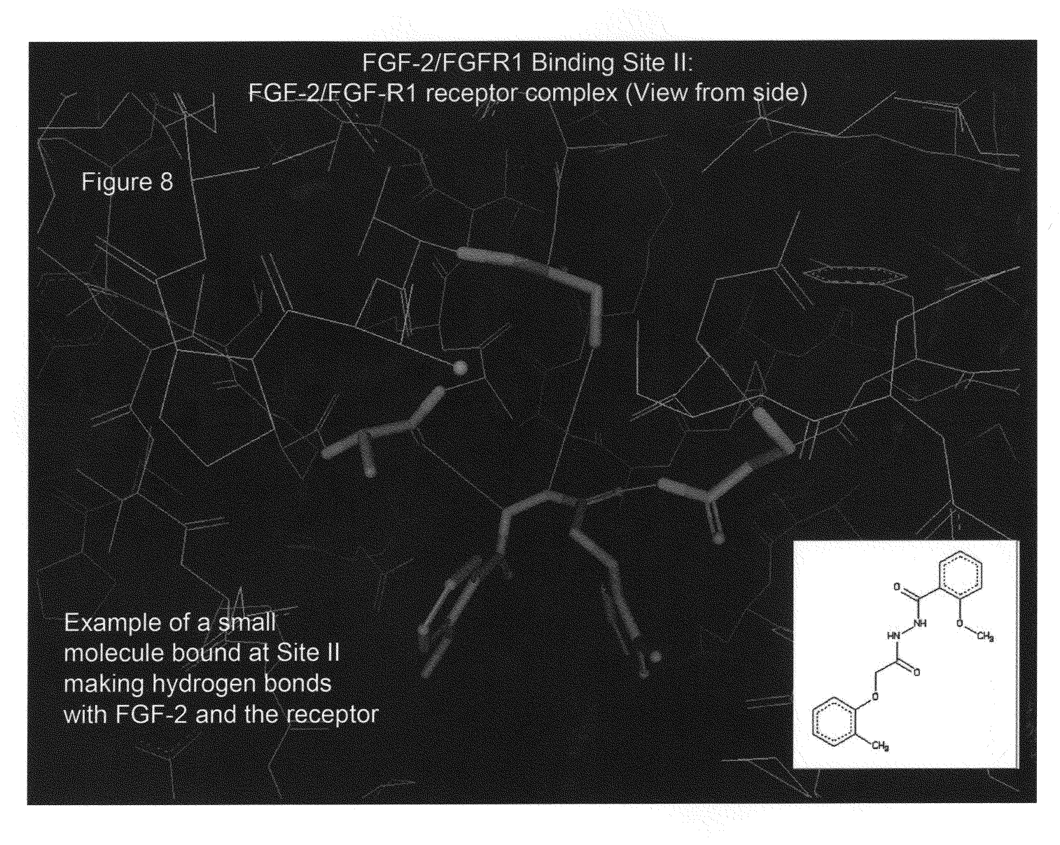

Compounds identified by this method are shown in FIG. 3 for Site I and in FIG. 4 for Site II. The docked structures of multiple compounds at Site I and Site II are shown in FIG. 5 and FIG. 6. As is shown in the figures, the organic molecules make extensive contacts with the receptor and the two molecules of FGF-2 on either side. FIG. 7 shows the hydrogen bond formation of an example compound with FGF-2 and FGF-R1 at Site I, and FIG. 8 shows the hydrogen bond formation of an example compound with FGF-2 and FGF-R1 at Site II.

Organic molecules identified by in silico or other methods may be further screened in an in vitro assay to examine upregulation of FGF-2 receptor signaling, e.g., in the presence of sub-maximal concentrations of FGF-2. An exemplary in vitro assay is provided below. Candidates identified by this assay may then be advanced to in vivo assays of particular disease, e.g., functional stroke recovery in rodents.

EPO

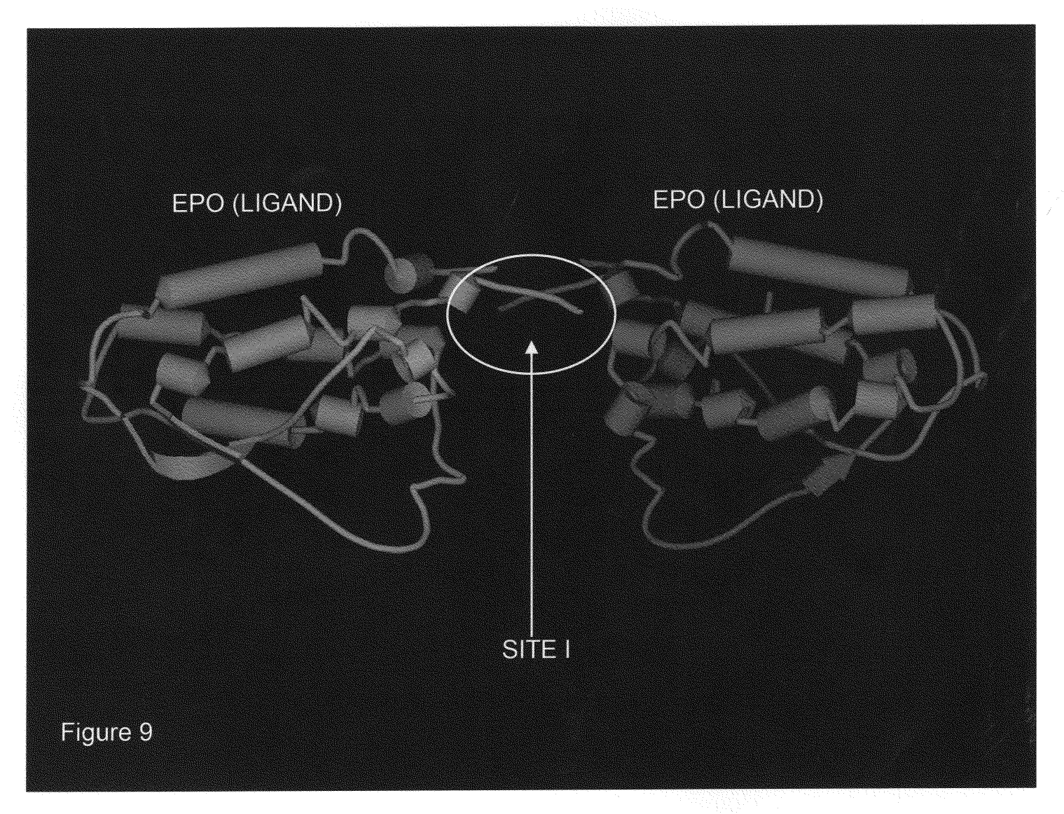

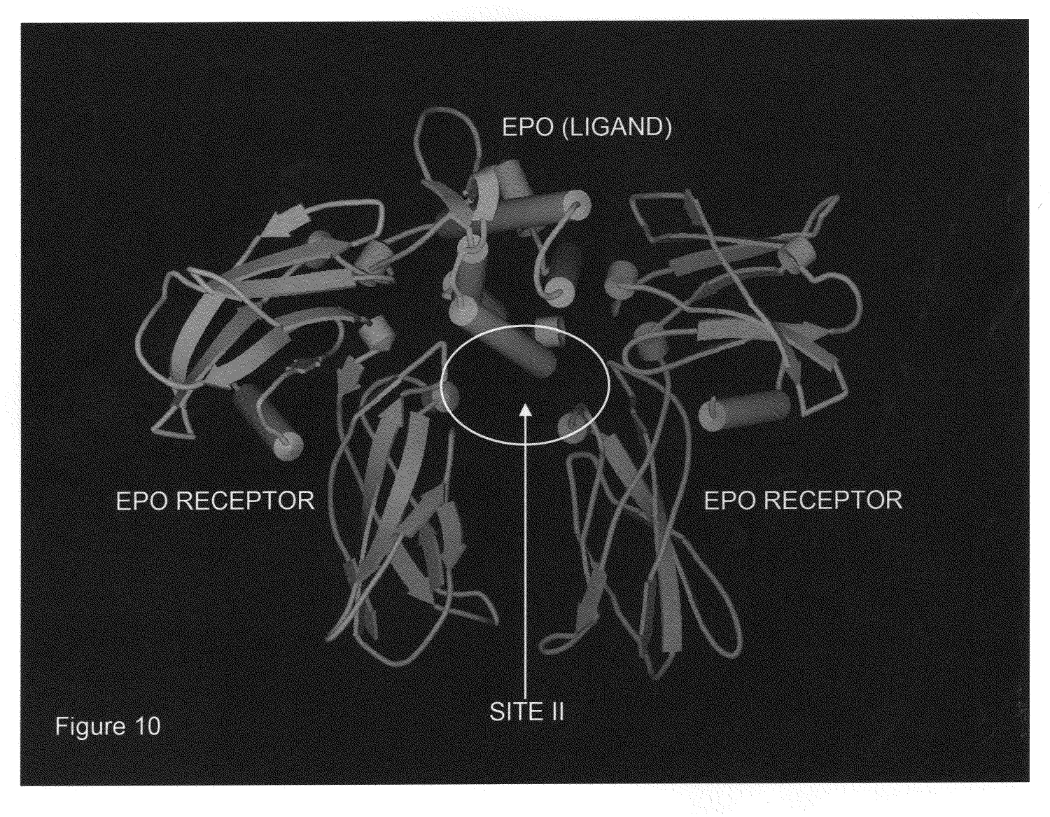

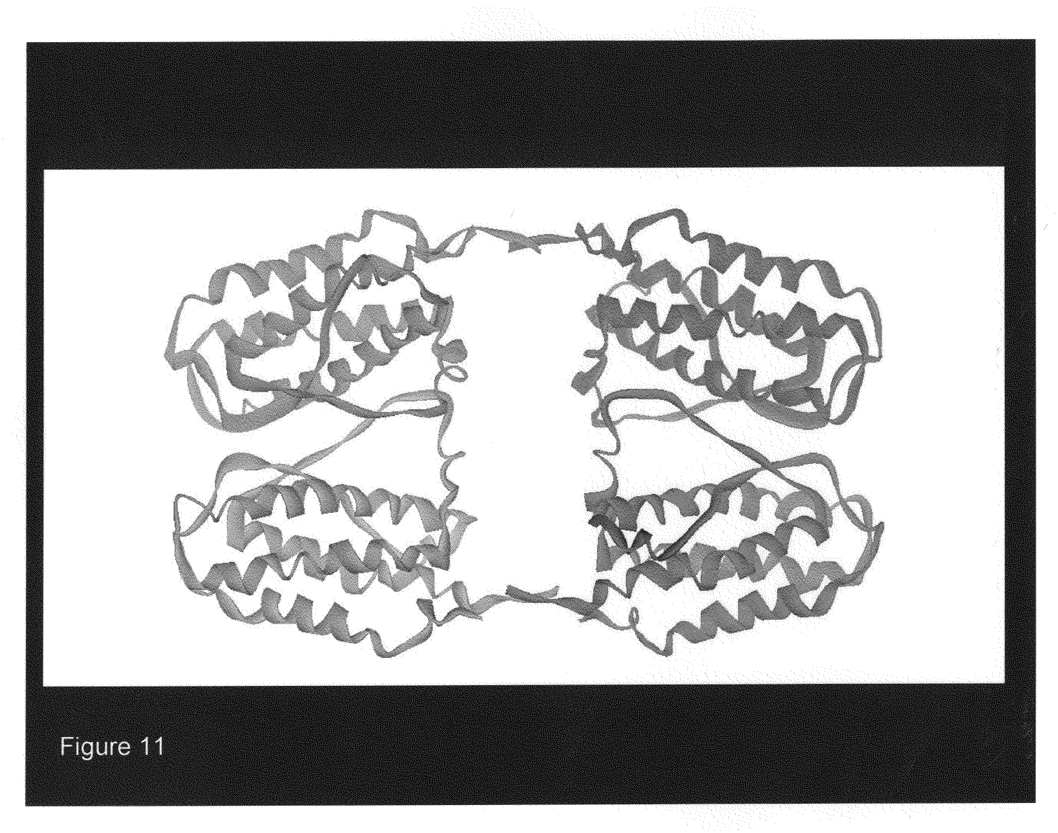

A similar strategy was applied to identify organic molecules to enhance binding between EPO and the EPO receptor (EPO-R). Molecular models of the EPO/EPO-R complex were constructed, based on the published crystal structure of this complex and experimental results from covalent dimers (FIGS. 9 and 10) (pdb code 1EER; Syed et al. Nature 395: 511-516 (1998)). These models identified two bindings sites for organic molecules. In addition, we identified two regions where EPO could form noncovalent dimers. Simultaneous dimerization at both of these regions results in formation of a noncovalent EPO tetramer (FIG. 11). Organic molecules binding to Site I encourage dimer/tetramer formation by binding simultaneously to two EPO molecules at their N—C terminal ends. For Site I (FIG. 9), the following amino acids to which an organic molecule may bind were identified: Ala-1, Pro-2, Pro-3, Arg-4, Leu-5, Ile-6, Cys-7, Asp-8, Cys-161, Arg-162, Thr-163, Gly-164, Asp-166, and Arg-167. An organic molecule of the invention preferably binds noncovalently to at least one of these residues on each of two EPO molecules. As is shown in FIG. 9, two organic molecules may each simultaneously bind to two molecules of EPO when a tetramer is formed.

For Site II (FIG. 10), the following amino acids to which an organic molecule may bind were identified: on EPO: Asp-8, Ser-9, Arg-10, Val-11, Leu-12, Glu-13, Arg-14, Tyr-15, Leu-16, Leu-17, Glu-18, Ala-19, Lys-20, Glu-21, Ala-22, Glu-23, and Lys-24; and on EPO-R: Thr-148, Pro-149, Met-150, Thr-151, Ser-152, His-153, Arg-154, Leu-175, Glu-176, Gly-177, and Arg-178. An organic molecule of the invention preferably binds noncovalently to at least one of these residues on each of an EPO molecule and an EPO-R. As is shown in FIG. 10, the organic molecule may simultaneously bind to one molecule of EPO and two EPO-R molecules.

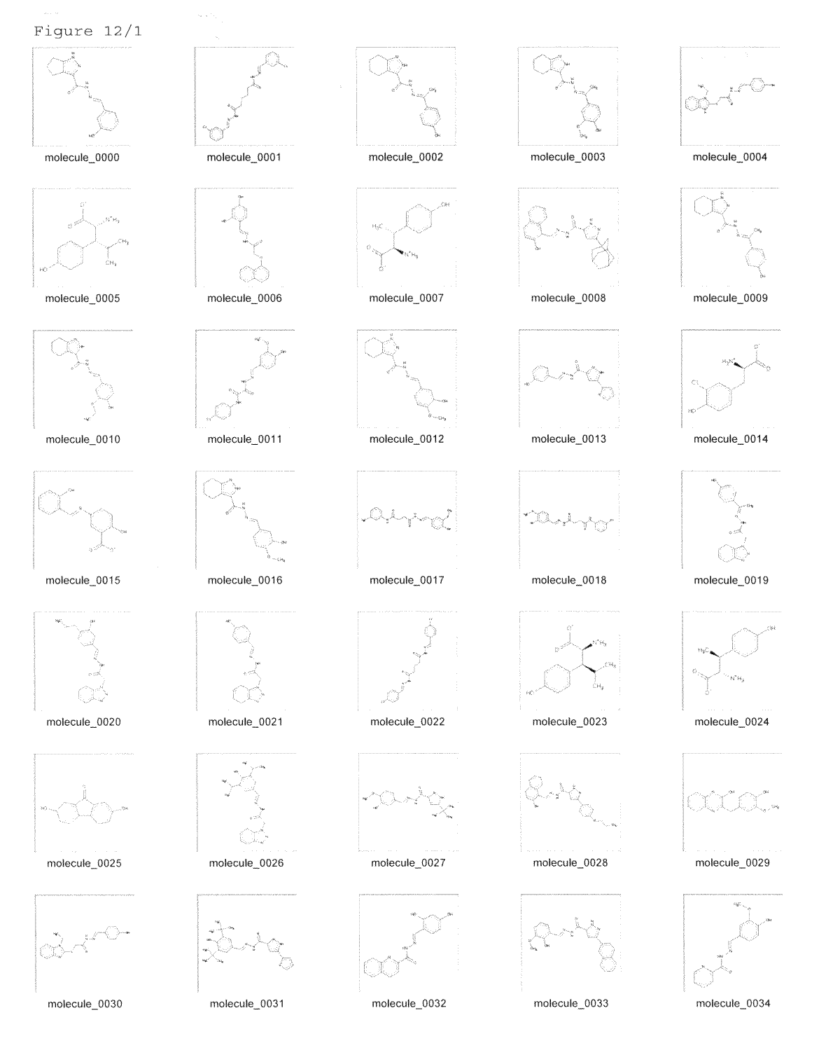

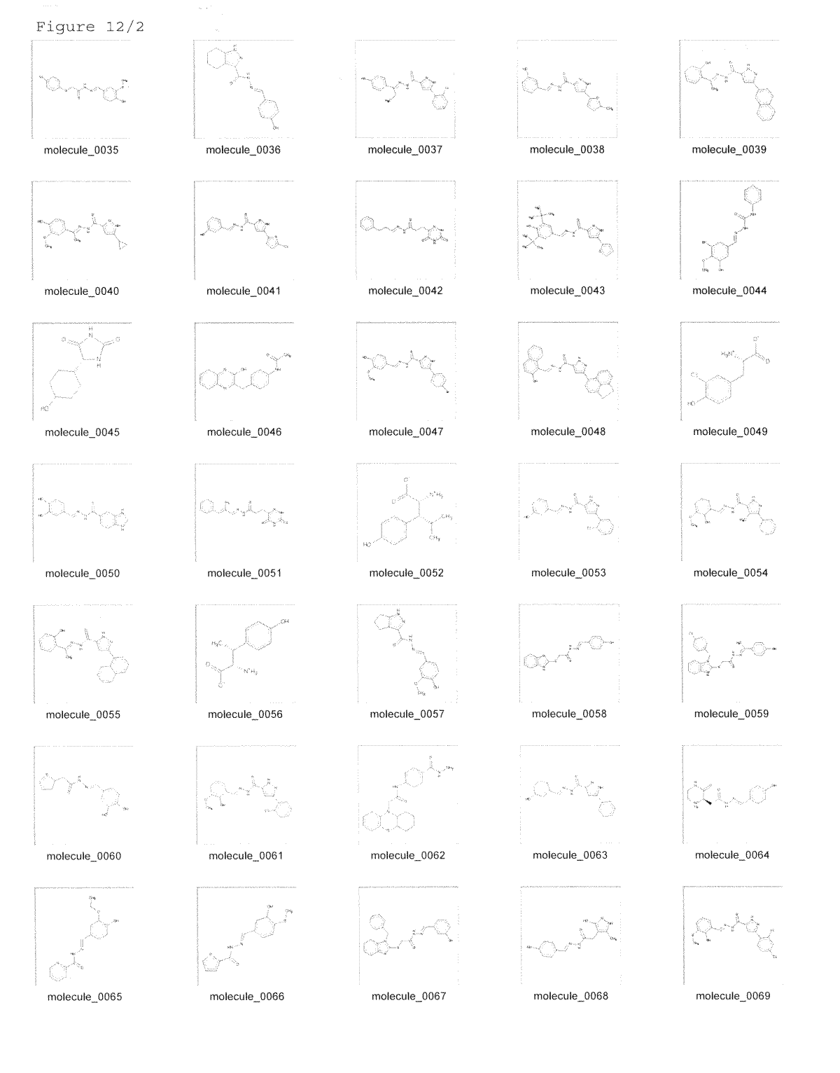

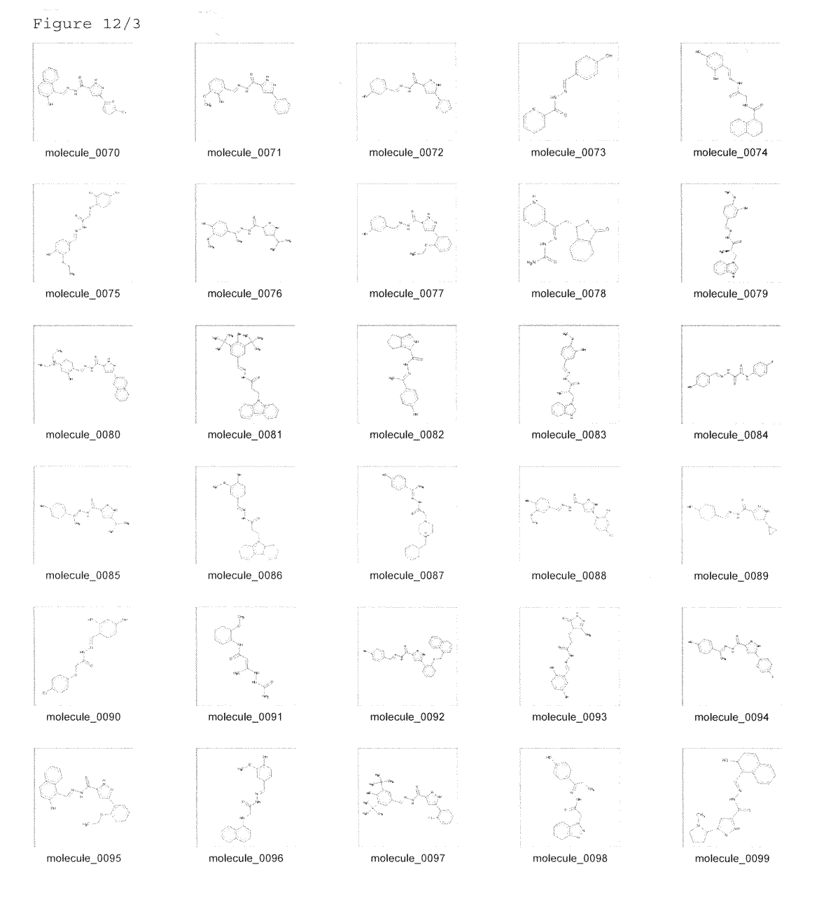

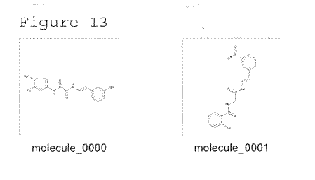

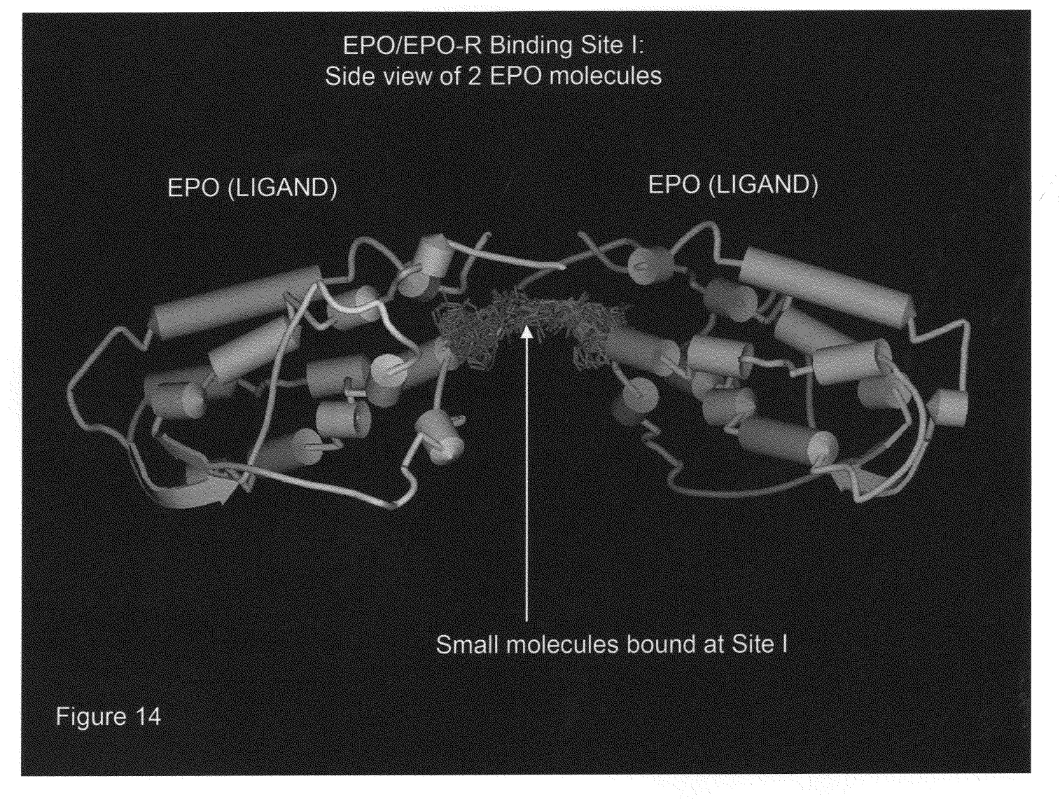

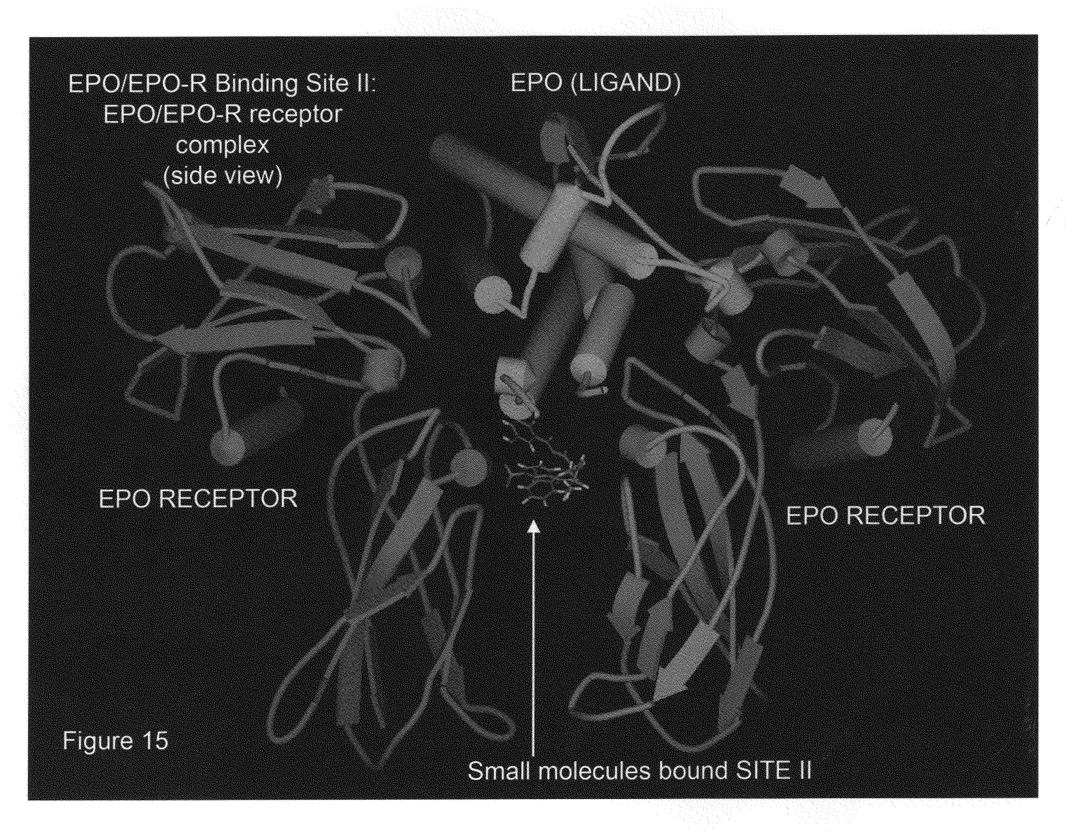

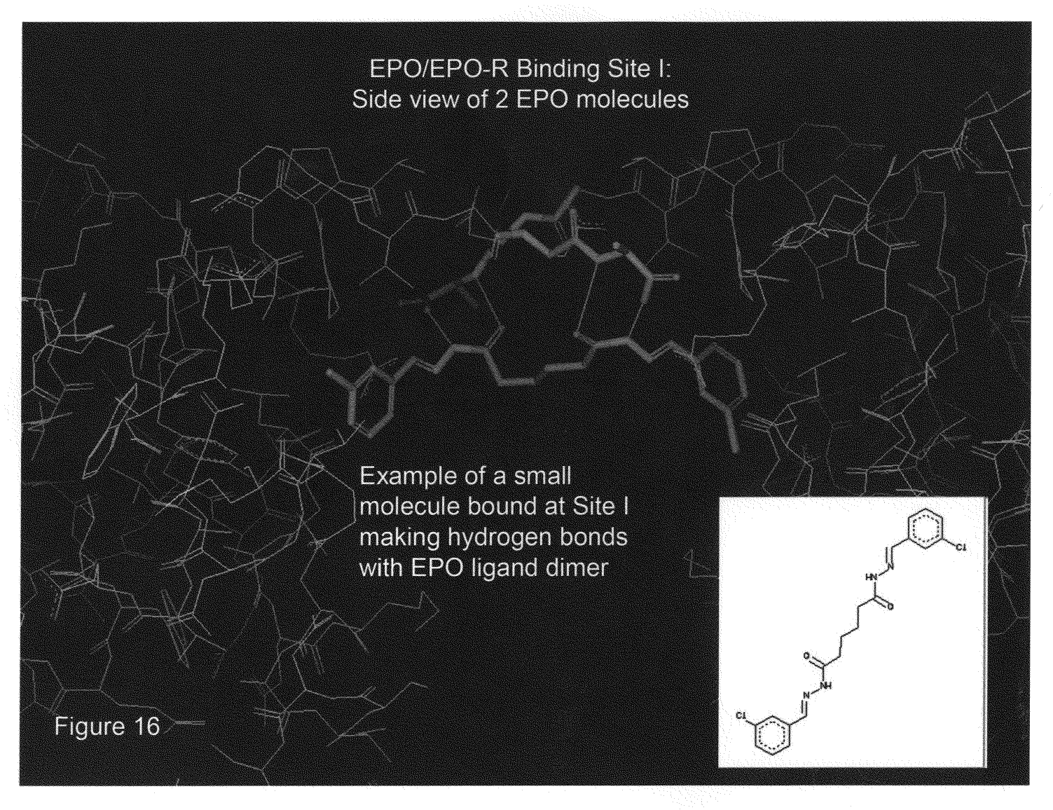

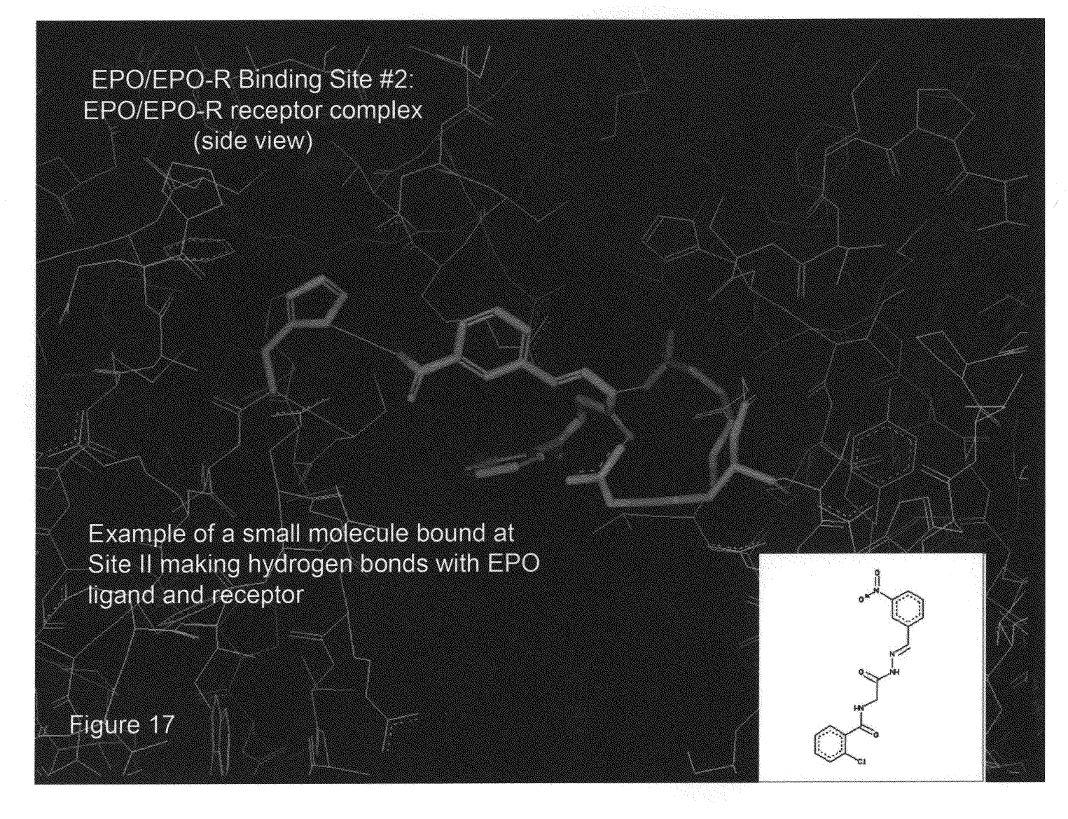

Compounds identified by this method are shown in FIG. 12 for Site I and in FIG. 13 for Site II. The docked structures of multiple compounds at Site I and Site II are shown in FIG. 14 and FIG. 15. As is shown in the figures, the organic molecules make extensive contacts with two molecules of EPO at Site I and EPO and two EPO-R molecules at Site II. FIG. 16 shows the hydrogen bond formation of an example compound with two EPO molecules at Site I, and FIG. 17 shows the hydrogen bond formation of an example compound with EPO and two EPO-R molecules at Site II.

Organic molecules identified by in silico or other methods may be further screened in an in vitro assay to examine upregulation of EPO receptor signaling, e.g., in the presence of sub-maximal concentrations of EPO. Candidates identified by this assay may then be advanced to in vivo assays of particular disease, e.g., anemia.

Therapeutic Methods

The organic molecules of the invention are, in general, suitable for any therapeutic use, in which increased receptor on-time (and thus decreased off-time), e.g., for FGF-2 and EPO, is desired. Organic molecules are typically easier to administer to a patient than protein ligands, e.g., FGF-2 or EPO. Accordingly, administration of the organic molecules of the invention provides an alternate route for upregulating the activity of a ligand-receptor complex by enhancing the binding of the ligand to the receptor.

Sources of organic molecules are well known in the art, including de novo synthesis, isolation or modification of naturally-occurring compounds, and selection from a library. Exemplary druglike properties for appropriate organic molecules are also well-known. Specific compounds may be identified using the in silico and/or in vitro methods described herein.

In principle, any medical disorder affected by enhancing the binding between a ligand and its receptor may be treated with the methods of the invention. FGF-2 has beneficial effects in cardiovascular, cerebrovascular, and peripheral vascular disease, including enhancement of functional recovery after stroke. Increasing the activity of FGF-2 by enhancing its receptor binding thus provides a treatment for cardiovascular, cerebrovascular, and peripheral vascular disease, including stroke recovery. EPO is administered to treat anemia having any of a number of causes, including chronic renal failure (whether or not associated with dialysis); HIV (e.g., in zidovudine-treated patients); and chemotherapy treatment of cancer. EPO is also administered to anemia patients undergoing surgery. Organic molecules of the invention that bind to EPO and/or its receptor may be employed to increase the activity of endogenous or exogenous EPO for all of these indications.

Organic molecules of the invention may also be employed in combinations with each other: e.g., as two or more organic molecules that target the same site or as two or more organic molecules that target different sites (e.g., Sites I and II for FGF-2 and EPO). Organic molecules may also be administered with or without other therapeutics for a particular condition. In particular, an organic molecule binding to Site I or II of FGF-2 or EPO may be administered together with exogenous FGF-2 or EPO, respectively, or within two hours of FGF-2 or EPO administration.

The dosages, timing, and routes of administration for a particular organic compound are determined by the skilled artisan based on the therapeutic, the particular disease, and the characteristics of the patient using standard techniques. Appropriate pharmaceutically acceptable carriers are also well known in the art.

### Exemplary In Vitro Assay for Upregulation of FGF-2 Signaling

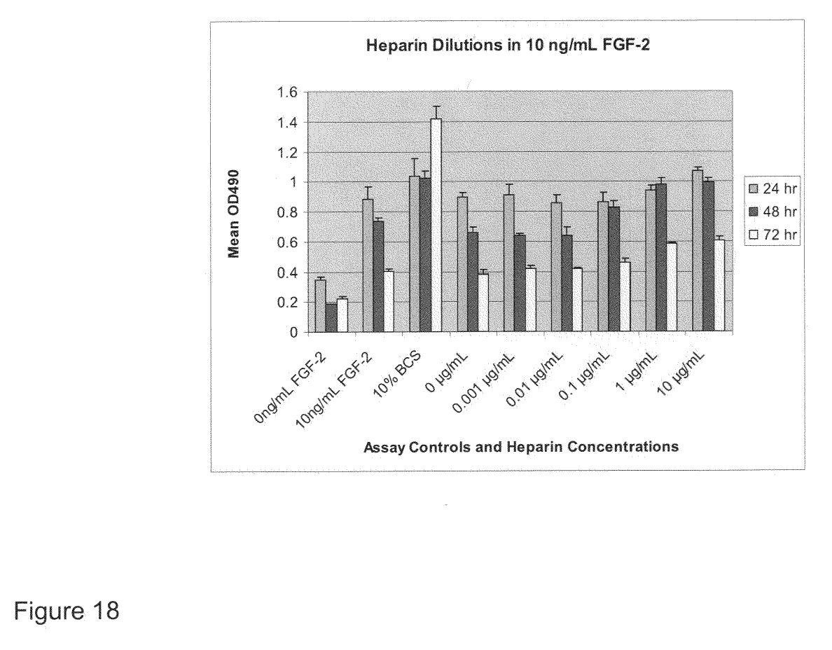

We developed a mitogenic assay on cells responsive to FGF-2. The assay is run in the presence of a submaximal concentration of FGF-2 (10 ng/mL). Heparin is a known potentiator of the activity of FGF-2, as FGF-2 activity is increased with increasing concentrations of heparin (FIG. 18). These results are quantified in Table 9. At 72 h of incubation in the presence of 10 ng/mL of FGF-2, the addition of 10 μg/mL of heparin increased mitogenic activity by 58%.

Organic compounds of the invention may be assayed for their effect on FGF-2 stimulation of growth using this assay. An increase in the amount of growth upon administration of an organic molecule of the invention indicates that it is a candidate compound for development as a therapeutic agent. For example, a “hit” can be defined as an increase in activity of >50%, in the presence of 10 ng/mL FGF-2 (with no added heparin).

The details of the assay are as follows.

Heparin Control Test

A. Cell Plating

1. Trypsinize cells. Remove a small aliquot of cell suspension for cell count and viability.

2. Count cells and measure cell viability using Cellometer.

2.1 Cell count: 7.57×10⁵ cells/mL.
2.2 Total cells: 7.57×10⁵ cells/mL×11.5 mL cell suspension=8.71×10⁶ cells.

### 2.3 Viability: 98.6%

2.4 Total cells: 8.71×10⁶×0.986=8.59×10⁶ viable cells.

3. Centrifuge trypsin/trypsin neutralizer/cell suspension at 250×g for 6 minutes to pellet cells. Aspirate supernatant.

3.1 Determine volume of DMEM/10% BCS/1% Pen-Strep (media) with which to resuspend cell pellet: 8,590,000 cells/62,500 cells/mL=137 mL media.
3.2 If necessary, make a 10× dilution by dividing the result from 3.1 by 10: 137 mL media/10=13.7 mL. Then add 3 mL of the 10× cell suspension to 27 mL of media.

4. Transfer 80 μL of cell suspension to the appropriate wells of three 96-well tissue culture-treated plates.

5. Incubate plates at 37° C./5% CO₂ for 18 hr.

B. Serum Starvation

Aspirate media from plates.

Rinse cells once with 100 μL/well 1×PBS.

Add 80 μL/well DMEM/0.1% BCS/1% Pen-Strep (0.1% BCS media), and incubate cells for 24 hr at 37° C./5% CO₂.

C. FGF-2 and Heparin Preparation

A mass of 20 mg of heparin was dissolved in 2.0 mL of 0.1% BCS media for a final concentration of 10 mg/mL.

Preparation of 10× Heparin Solution: The 10 mg/mL heparin solution was diluted to 100 μg/mL by adding 50 μL of the 10 mg/mL solution to 4,950 μL of 0.1% BCS media.

A 1:10 intermediate dilution was prepared by adding 10 μL of 100 μg/mL FGF-2 stock to 90 μL of 1×PBS/0.5% BSA, for a final concentration of 10 μg/mL.

Preparation of 10×FGF-2: The 1:10 intermediate was further diluted by adding 50 μL to 4,950 μL of 0.1% BCS media, for a final concentration of 100 ng/mL. The 100 ng/mL FGF-2 solution was diluted by adding 500 μL to 4,500 μL of 0.1% BCS media, for a final concentration of 10 ng/mL.

Preparation of 10× Heparin Dilutions: Prepare serial dilutions of 10× heparin in media containing 0.1% BCS as shown in Table 2.

TABLE 2
10X Heparin Preparation

| 10X Heparin | Vol 10X Heparin | Vol 0.1 BCS media | Total Vol |
|---|---|---|---|
| ug/ml | (ul) | (ul) | (ul) |
| 10 | 2000 | 0 | 2000 |
| 1 | 200 ul of 10 ug/ml | 1800 | 2000 |
| 0.1 | 200 ul of 1 ug/ml | 1800 | 2000 |
| 0.01 | 200 ul of 0.1 ug/ml | 1800 | 2000 |
| 0.001 | 200 ul of 0.01 ug/ml | 1800 | 2000 |
| 0 | 0 ul | 2000 | 2000 |

6. 5× Heparin/FGF-2 Dilutions: Mix equal volumes of 10×FGF-2 (Steps 3 and 4) and 10× heparin stocks (Step 5) to create the 5× Treatment Intermediates as shown in Tables 3 and 4.

TABLE 3
Heparin Dilutions in 1 ng/mL FGF-2

| 10X Heparin | 10X FGF-2 [1 ng/ml] | Total Vol (ul) |
|---|---|---|
| 200 ul of 10 ug/ml | 200 ul | 400 |
| 200 ul of 1 ug/ml | 200 ul | 400 |
| 200 ul of 0.1 ug/ml | 200 ul | 400 |
| 200 ul of 0.01 ug/ml | 200 ul | 400 |
| 200 ul of 0.001 ug/ml | 200 ul | 400 |
| 200 ul of 0 ug/ml | 200 ul | 400 |

For 1 ng/ml FGF-2 - Use 10X FGF-2 (1 ng/ml) Stock

TABLE 4
Heparin Dilutions in 10 ng/mL FGF-2

| 10X Heparin | 10X FGF-2 [10 ng/ml] | Total Vol (ul) |
|---|---|---|
| 200 ul of 10 uq/ml | 200 ul | 400 |
| 200 ul of 1 ug/ml | 200 ul | 400 |
| 200 ul of 0.1 uq/ml | 200 ul | 400 |
| 200 ul of 0.01 ug/ml | 200 ul | 400 |
| 200 ul of 0.001 uq/ml | 200 ul | 400 |
| 200 ul of 0 ug/ml | 200 ul | 400 |

For 10 ng/ml FGF-2 - Use 10X FGF-2 [10 ng/ml] Stock

D. Cell Titer AQueous One Development

Add 20 μL of CellTiter AQueous One Solution to each of the treated wells.

Incubate for 1 hr at 37° C./5% CO₂.

Read plate at 490 nm.

Results of these assays are shown in the following tables.

TABLE 5
Assay Controls - Average O.D. at 490 nm, Standard Deviation and CV %

| | 24 hr | | | 48 hr | | | 72 hr | | |
|---|---|---|---|---|---|---|---|---|---|
| | Mean | Std | CV | Mean | Std | CV | Mean | Std | CV |
| Controls | OD490 | Dev | % | OD490 | Dev | % | OD490 | Dev | % |
| 0 ng/mL FGF-2 | 0.349 | 0.023 | 6.6 | 0.186 | 0.005 | 2.9 | 0.222 | 0.012 | 5.2 |
| 10 ng/mL FGF-2 | 0.884 | 0.085 | 9.6 | 0.737 | 0.023 | 3.1 | 0.401 | 0.014 | 3.6 |
| 10% BCS | 1.035 | 0.117 | 11.3 | 1.022 | 0.048 | 4.7 | 1.418 | 0.083 | 5.8 |

TABLE 6
Heparin and 1 ng/mL FGF-2 Stimulation of NIH/3T3
Cells -Mean OD490, Standard Deviation and CV %

| | | 1 ng/mL FGF-2 | | | | | | | | |
|---|---|---|---|---|---|---|---|---|---|---|
| | | 24 hr | | | 48 hr | | | 72 hr | | |
| Heparin | | Mean | Std | CV | Mean | Std | CV | Mean | Std | CV |
| (μg/mL) | | OD490 | Dev | % | OD490 | Dev | % | OD490 | Dev | % |
| 0 | μg/mL | 0.465 | 0.019 | 4.2 | 0.197 | 0.012 | 6.3 | 0.199 | 0.017 | 8.7 |
| 0.001 | μg/mL | 0.477 | 0.030 | 6.3 | 0.222 | 0.016 | 7.3 | 0.208 | 0.022 | 10.7 |
| 0.01 | μg/mL | 0.430 | 0.025 | 5.7 | 0.200 | 0.008 | 4.1 | 0.199 | 0.015 | 7.7 |
| 0.1 | μg/mL | 0.419 | 0.009 | 2.1 | 0.226 | 0.010 | 4.5 | 0.199 | 0.016 | 8.0 |
| 1 | μg/mL | 0.398 | 0.016 | 4.0 | 0.202 | 0.013 | 6.5 | 0.212 | 0.011 | 5.2 |
| 10 | μg/mL | 0.433 | 0.012 | 2.7 | 0.196 | 0.008 | 4.0 | 0.188 | 0.010 | 5.1 |

TABLE 7
Heparin and 1 ng/mL FGF-2 Stimulation of NIH/3T3
Cells -- Percent Difference from Mean OD490
of 0 μg/mL Heparin Concentration

| | | 1 ng/mL FGF-2 | | |
|---|---|---|---|---|
| Heparin | | Percent Difference from 0 μg/mL Heparin Mean OD490 | | |
| (μg/mL) | | 24 hr | 48 hr | 72 hr |
| 0 | μg/mL | 0.0% | 0.0% | 0.0% |
| 0.001 | μg/mL | 2.6% | 12.7% | 4.5% |
| 0.01 | μg/mL | −7.5% | 1.5% | 0.0% |
| 0.1 | μg/mL | −9.9% | 14.7% | 0.0% |
| 1 | μg/mL | −14.4% | 2.5% | 6.5% |
| 10 | μg/mL | −6.9% | −0.5% | −5.5% |

TABLE 8
Heparin and 10 ng/mL FGF-2 Stimulation of NIH/3T3
Cells - Mean OD490, Standard Deviation and CV %

| | | 10 ng/mL FGF-2 | | | | | | | | |
|---|---|---|---|---|---|---|---|---|---|---|
| | | 24 hr | | | 48 hr | | | 72 hr | | |
| Heparin | | Mean | Std | CV | Mean | Std | CV | Mean | Std | CV |
| (μg/mL) | | OD490 | Dev | % | OD490 | Dev | % | OD490 | Dev | % |
| 0 | μg/mL | 0.898 | 0.024 | 2.6 | 0.661 | 0.032 | 4.8 | 0.381 | 0.027 | 7.0 |
| 0.001 | μg/mL | 0.912 | 0.066 | 7.2 | 0.641 | 0.011 | 1.6 | 0.416 | 0.022 | 5.2 |
| 0.01 | μg/mL | 0.859 | 0.055 | 6.4 | 0.641 | 0.056 | 8.7 | 0.417 | 0.010 | 2.3 |
| 0.1 | μg/mL | 0.863 | 0.064 | 7.4 | 0.830 | 0.041 | 5.0 | 0.459 | 0.029 | 6.2 |
| 1 | μg/mL | 0.938 | 0.033 | 3.5 | 0.978 | 0.048 | 4.9 | 0.583 | 0.008 | 1.4 |
| 10 | μg/mL | 1.071 | 0.019 | 1.8 | 0.992 | 0.034 | 3.4 | 0.602 | 0.030 | 4.9 |

TABLE 9
Heparin and 10 ng/mL FGF-2 Stimulation of NIH/3T3
Cells -- Percent Difference from Mean OD490
of 0 μg/mL Heparin Concentration

| | | 10 ng/mL FGF-2 | | |
|---|---|---|---|---|
| Heparin | | Percent Difference from 0 μg/mL Heparin OD490 | | |
| (μg/mL) | | 24 hr | 48 hr | 72 hr |
| 0 | μg/mL | 0.0% | 0.0% | 0.0% |
| 0.001 | μg/mL | 1.6% | −3.0% | 9.2% |
| 0.01 | μg/mL | −4.3% | −3.0% | 9.4% |
| 0.1 | μg/mL | −3.9% | 25.6% | 20.5% |
| 1 | μg/mL | 4.5% | 48.0% | 53.0% |
| 10 | μg/mL | 19.3% | 50.1% | 58.0% |

### Other Embodiments

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

What is claimed is:

1. A method of enhancing the binding of a first ligand to a cellular receptor, said method comprising the steps of contacting said ligand with an exogenous organic molecule that non-covalently binds to said first ligand and non-covalently binds to either (i) a second ligand for said receptor or (ii) said receptor, thereby enhancing the binding of said first ligand to said receptor.

2. The method of claim 1, wherein said organic molecule noncovalently binds to said second ligand for said receptor.

3. The method of claim 1, wherein said organic molecule noncovalently binds to said receptor.

4. The method of claim 1, wherein said organic molecule noncovalently binds to said second ligand and said receptor.

5. The method of claim 1, wherein said organic molecule noncovalently binds to two molecules of said receptor.

6. The method of claim 1, wherein said first ligand is FGF-2 or EPO.

7. The method of claim 6, wherein said organic molecule noncovalently binds to one of Asn-27, Arg-120, Thr-121, Lys-125, Lys-129, Gln-134, Lys-135, and Ala-136 of FGF-2; and one of Glu-159, Lys-160, Lys-163, Lys-172, Thr-173, Phe-176, Lys-177, Lys-207, Val-208, Arg-209, Thr-212, Ile-216, Met-217, Asp-218, and Ser-219 of FGF-R1.

8. The method of claim 6, wherein said organic molecule noncovalently binds to one of Arg-97, Leu-98, Glu-99, Ser-100, Asn-101, and Asn-102 of FGF-2; and one of Pro-169, Ala-170, Ala-171, Asp-217, Ser-218, Val-219, Val-220, Pro-221, Ser-222, Asp-223, Val-248, Glu-249, Arg-250, and Ser-251 of FGF-R 1.

9. The method of claim 6, wherein said organic molecule noncovalently binds to one of Ala-1, Pro-2, Pro-3, Arg-4, Leu-5, Ile-6, Cys-7, Asp-8, Cys-161, Arg-162, Thr-163, Gly-164, Asp-166, and Arg-167 in each of two molecules of EPO.

10. The method of claim 6, wherein said organic molecule noncovalently binds to one of Thr-148, Pro-149, Met-150, Thr-151, Ser-152, His-153, Arg-154, Leu-175, Glu-176, Gly-177, and Arg-178 of EPO-R; and one of Asp-8, Ser-9, Arg-10, Val-11, Leu-12, Glu-13, Arg-14, Tyr-15, Leu-16, Leu-17, Glu-18, Ala-19, Lys-20, Glu-21, Ala-22, Glu-23, and Lys-24 of EPO.

11. A pharmaceutical composition comprising (a) a first ligand for a cellular receptor and/or (b) an organic molecule that non-covalently binds to said ligand and either (i) a second ligand for said receptor or (ii) said receptor to enhance the binding of said first ligand to said receptor; and (c) a pharmaceutically acceptable carrier.

12. The composition of claim 11, wherein the first ligand is FGF-2 or EPO.

13. A tetramer comprising four, noncovalently bound molecules of EPO, wherein each EPO molecule is noncovalently bound to two other EPO molecules.

14. The tetramer of claim 13, further comprising two exogenous organic molecules that stabilize the formation of noncovalent bonds between two or more EPO molecules.

15. The tetramer of claim 13, wherein two molecules of EPO are noncovalently bound via Ala-1, Pro-2, Pro-3, Arg-4, Leu-5, Ile-6, Cys-7, Asp-8, Cys-161, Arg-162, Thr-163, Gly-164, Asp-166, and Arg-167.

16. The tetramer of claim 14, wherein each of said organic molecules noncovalently binds to one of Ala-1, Pro-2, Pro-3, Arg-4, Leu-5, Ile-6, Cys-7, Asp-8, Cys-161, Arg-162, Thr-163, Gly-164, Asp-166, and Arg-167 in each of two molecules of EPO.

17. A pharmaceutical composition comprising a pharmaceutically effective amount of a compound of FIG. 3, 4, 12, or 13, together with a pharmaceutically acceptable carrier.

18. A method of treating a medical condition in a human patient, said method comprising administering to said patient, in an amount sufficient to treat said medical condition, an organic compound that non-covalently binds to a first ligand for a cellular receptor and non-covalently binds to either (i) a second ligand for said receptor or (ii) said receptor to enhance the binding of said first ligand to said receptor, thereby treating said medical condition.

19. The method of claim 18, wherein said first ligand is FGF-2 or EPO.

20. The method of claim 18, wherein said medical condition is anemia.

21. The method of claim 18, wherein said medical condition is cerebrovascular, cardiovascular, or peripheral vascular disease.

22. The method of claim 21, wherein said cerebrovascular disease is stroke.

23. The method of claim 19, wherein said organic molecule noncovalently binds to one of Asn-27, Arg-120, Thr-121, Lys-125, Lys-129, Gln-134, Lys-135, and Ala-136 of FGF-2; and one of Glu-159, Lys-160, Lys-163, Lys-172, Thr-173, Phe-176, Lys-177, Lys-207, Val-208, Arg-209, Thr-212, Ile-216, Met-217, Asp-218, and Ser-219 of FGF-R1.

24. The method of claim 19, wherein said organic molecule noncovalently binds to one of Arg-97, Leu-98, Glu-99, Ser-100, Asn-101, and Asn-102 of FGF-2; and one of Pro-169, Ala-170, Ala-171, Asp-217, Ser-218, Val-219, Val-220, Pro-221, Ser-222, Asp-223, Val-248, Glu-249, Arg-250, and Ser-251 of FGF-R1.

25. The method of claim 19, wherein said organic molecule noncovalently binds to one of Ala-1, Pro-2, Pro-3, Arg-4, Leu-5, Ile-6, Cys-7, Asp-8, Cys-161, Arg-162, Thr-163, Gly-164, Asp-166, and Arg-167 in each of two molecules of EPO.

26. The method of claim 19, wherein said organic molecule noncovalently binds to one of Thr-148, Pro-149, Met-150, Thr-151, Ser-152, His-153, Arg-154, Leu-175, Glu-176, Gly-177, and Arg-178 of EPO-R; and one of Asp-8, Ser-9, Arg-10, Val-11, Leu-12, Glu-13, Arg-14, Tyr-15, Leu-16, Leu-17, Glu-18, Ala-19, Lys-20, Glu-21, Ala-22, Glu-23, and Lys-24 of EPO.
